(19) United States
(12) Patent Application Publication
(10) Pub. No.: US 2020/0138620 A1
(43) Pub. Date: May 7, 2020

(54) Foldable Urination Device

(71) Applicant: Mountain Group, LLC, East Chatham, NY (US)

(72) Inventor: Brian Schwab, East Chatham, NY (US)

(73) Assignee: Mountain Group, LLC, East Chatham, NY (US)

(21) Appl. No.: 16/677,602

(22) Filed: Nov. 7, 2019

Related U.S. Application Data
(60) Provisional application No. 62/757,142, filed on Nov. 7, 2018.
(60) Provisional application No. 62/757,140, filed on Nov. 7, 2018.

(51) Int. Cl. A61F 5/455; A61F 5/453

(52) U.S. Cl. CPC A61F 5/4556; A61F 5/453

(57) ABSTRACT

A foldable urination device comprises a body having left and right curved fold lines and left and right side fold lines. Left and right long tabs are connected to respective left and right sides of the body. The left curved fold line and the left side fold lines are disposed on the left side of the body, and the right curved fold line and the right side fold line are disposed on a right side of the body, wherein the central fold line is disposed between the left and right curved fold lines and side fold lines. When the foldable urination device is folded along the curved and side fold lines, the foldable urination device forms a curved passageway having an entrance and an exit. The curved fold line has greater curvature on a top part of the body and lesser curvature on a bottom part of the body. The left long tab is adhered to the right side of the body. A folded handle is attached to the left long tab.

### TECHNICAL FIELD

The present invention, in some embodiments thereof, generally relate to urination devices.

### BACKGROUND OF THE INVENTION

Conventionally, there are assistive urination devices for female use. There are few urination devices for men. Some men have difficulty in urine discharging (urinating) because of buried penis, hidden penis, penis shrinkage, benign prostatic hypertrophy, cancer of the prostate/bladder, urinary blockage or neurogenic bladder dysfunction.

Therefore, there is a need for a urination device to assist a male user to urinate easier and prevent urine splashback.

### BRIEF SUMMARY OF EMBODIMENTS OF THE INVENTION

The present invention generally relates to a urination device and a method of using the urination device. The urination device includes a first end having a first opening, a second end having a second opening, a funnel shaped body, one or more flaps, and one or more of foldaway handles. The urination device can be placed onto a male user's penis or perineum so as to assist the male user urinate easily. In addition, the urination device is collapsible for compact storage and preferably made of flushable paper or the like for easy disposability.

In one embodiment, a first opening, a first ring-shaped element positioned close to the first opening and one or more second ring-shaped elements, wherein the first ring-shaped element and the one or more second ring-shaped elements are adapted to be stacked concentrically and stored in a packaged form and extended to an expanded form, and wherein the first ring-shaped element is larger in size than the one or more second ring-shaped elements and is located in the outer most location when stacked concentrically.

In another embodiment, a second opening smaller than the first opening; one or more flaps forming into the first opening is positioned; one or more handles attached to the first ring-shaped element so that a user of the urination device is able to hold the urination device via the one or more handles for directing a urine liquid away from the user while the user is using the urination device.

In still another embodiment, the urination device includes a rim connected to the body and the one or more flaps, and one or more handles are adapted to be folded together to be held by the user and unfolded to be stored.

In yet another embodiment, the urination device includes the body of the urination device is adapted to be folded in at least half of its original size using one or more folding lines as guides, and the one or more flaps is capable of being folded into a packed storage position and being extended in use in an extended shape.

In another embodiment, the urination device includes the first opening is adapted to receive a urine liquid of the user, and the urine liquid is directed to flow through the body of the urination device and out of the urination device through the second opening to a target location.

In yet another embodiment, the urination device includes a first end having a first opening, a second end having a second opening, a funnel shaped body, one or more flaps, and one or more of foldaway handles. The urination device can be placed onto a male user's penis or perineum so as to assist the male user urinate easily. In addition, the urination device is foldable for compact storage and preferably made of flushable paper or the like for easy disposability.

In one embodiment, a body being shaped in a rounded funnel shape when the body is unfolded and expanded in use, the body includes a first opening, one or more flaps forming into the first opening, and a second opening smaller than the first opening, and one or more handles attached to a portion of the body close to the first opening so that a user of the urination device is able to hold the urination device via the one or more handles for directing a urine liquid away from the user while the user is using the urination device.

In another embodiment, the one or more handles are adapted to be folded together to be held by the user and unfolded to be stored, and the body is adapted to be folded in at least half of its original size using one or more folding lines as guides.

In still another embodiment, body is adapted to be folded so as to be in a flat storage position, and the body is adapted to be unfolded and expanded into a rounded funnel shape by using the one or more handles and one or more folding lines as guides.

In yet another embodiment, wherein the one or more flaps is capable of being folded into a packed storage position and being extended in use in an extended shape, and the first opening is adapted to receive an urine liquid of the user, and the urine liquid is directed to flow through the body of the urination device and out of the urination device through the second opening to a target location.

In another embodiment, a body being shaped in a rounded funnel shape when the body is unfolded and expanded in use, the body includes a first opening, one or more flaps forming into the first opening; and a second opening smaller than the first opening; and one or more handles being attached to a portion of the body close to the first opening and adapted to direct a urine liquid away from a user while the user is using the urination device, wherein the one or more handles are adapted to be folded together to be held by the user and unfolded to be stored. Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

In one embodiment, a foldable urination device comprises a body having a central fold line, left and right curved fold lines and a left side fold line and a left long tab connected to a left side of the body along the left side fold line. The left curved fold line and the left side fold lines are disposed on the left side of the body, and the right curved fold line, wherein the central fold line is disposed between the left and right curved fold lines. When the foldable urination device is folded along the curved and side fold lines, the foldable urination device forms a curved passageway having an entrance and an exit. A user of the urination device is able to hold the urination device directing a urine stream away from the user.

In another embodiment, a right fold line is disposed on a right side of the body and a right long tab connected to the right side of the body along the right fold line. The urination device comprises a flat sheet and the fold lines are scored lines. The urination device has top, bottom, left and right perimeter edges, wherein the top perimeter edge is an arcuate edge.

In a further embodiment, the urination device has short tabs attached to the bottom perimeter edge. In a folded state, the device comprises at least three sides, at least one of which is curved. Optionally the devices has four edges having at least one curved edge. In a folded state, an entrance is formed that is larger than the exit.

It yet another embodiment, the urination device has a minor tab connected to the left long tab. When the urination device is in a folded state, the left long tab is adhered to the right side of the body. A handle is attached to one of the long tabs and the long tab is folded along a side fold line. The left and right long tabs are attached to each other at their surfaces and a surface of a short tab is adhered to the body.

In still a further embodiment, the left and right curved fold lines and the left and right side fold lines are symmetrically disposed on either side of the central fold line.

In one embodiment, a foldable urination device comprises a body having left and right curved fold lines and left and right side fold lines. Left and right long tabs are connected to respective left and right sides of the body. The left curved fold line and the left side fold lines are disposed on the left side of the body, and the right curved fold line and the right side fold line are disposed on a right side of the body. When the foldable urination device is folded along the curved and side fold lines, the foldable urination device forms a curved passageway having an entrance and an exit.

In another embodiment, the foldable urination device is folded and the left and right tabs are adhered to each other. A central fold line is disposed down a middle of the body. A handle is attached to one of the tabs and wherein the device is folded into two flat layers resting against each other.

In a further embodiment, a foldable urination device comprises a body having left and right curved fold lines and left and right side fold lines. Left and right long tabs are connected to respective left and right sides of the body. The left curved fold line and the left side fold lines are disposed on the left side of the body, and the right curved fold line and the right side fold line are disposed on a right side of the body, wherein the central fold line is disposed between the left and right curved fold lines and side fold lines. When the foldable urination device is folded along the curved and side fold lines, the foldable urination device forms a curved passageway having an entrance and an exit. The curved fold line has greater curvature on a top part of the body and lesser curvature on a bottom part of the body. The left long tab is adhered to the right side of the body. A folded handle is attached to the left long tab.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

Some of the figures included herein illustrate various embodiments of the invention from different viewing angles. Although the accompanying descriptive text may refer to such views as “top,” “bottom” or “side” views, such references are merely descriptive and do not imply or require that the invention be implemented or used in a particular spatial orientation unless explicitly stated otherwise.

FIG. 1A illustrates a perspective view of one example of a disposable and foldable male urination device in an expanded position according to one embodiment of the invention.

FIG. 1B illustrates a perspective view of another example of the disposable and foldable male urination device in a packaged position according to another embodiment of the invention.

FIG. 2A illustrates a side view of one example of a disposable and foldable male urination device of FIG. 1B in packaged position according to one embodiment of the invention.

FIG. 2B illustrates a front view of one example of a disposable and foldable male urination device of FIG. 1B in unpackaged position, showing one or more flaps of FIG. 1B unfolded according to one embodiment of the invention.

FIG. 2C illustrates a front view of one example of a disposable and foldable male urination device of FIG. 1B in unpackaged position, showing one or more flaps of FIG. 1B unfolded according to one embodiment of the invention.

FIG. 3A illustrates a perspective view of one example of the disposable and foldable male urination device of FIG. 1B in unpackaged position, showing one or more flaps of FIG. 1B unfolded according to one embodiment of the invention.

FIG. 3B illustrates a perspective view of another example of the disposable and foldable male urination device of FIG. 1B in unpackaged position, showing a flap of FIG. 1B unfolded according to another embodiment of the invention.

FIG. 3C illustrates a perspective view of another example of the disposable and foldable male urination device of FIG. 1A in a flattened position, showing one or more handles of FIG. 1A unfolded according to another embodiment of the invention.

FIG. 4A illustrates a right side view of one example of a disposable and foldable male urination device of FIG. 1A in a flattened position, showing a handle of FIG. 1A folding up according to one embodiment of the invention.

FIG. 4B illustrates a perspective view of one example of a disposable and foldable male urination device of FIG. 1A in a flattened position, showing a handle of FIG. 1A folding up according to one embodiment of the invention.

FIG. 5 illustrates a perspective view of one example of a disposable and foldable male urination device of FIG. 1A in a flattened position, showing the top part of the funnel of FIG. 1 folding back according to one embodiment of the invention.

FIG. 6 is a flowchart of one example of a method of operating a urination device having one or more flaps and one or more handles according to one embodiment of the invention. The figures are not intended to be exhaustive or to limit the invention to the precise form disclosed. It should be understood that the invention can be practiced with modification and alteration, and that the invention be limited only by the claims and the equivalents thereof.

FIG. 7A is a perspective view one example of a foldable urination device in an expanded position according to one embodiment of the invention.

FIG. 7B is a perspective view of another example of the foldable urination device in an expanded position according to another embodiment of the invention.

FIG. 8 is a perspective view of one example of a foldable urination device of FIG. 1A in packaged position according to one embodiment of the invention;

FIG. 9A is a top view of one example of the foldable urination device of FIG. 1A in unpackaged position according to one embodiment of the invention;

FIG. 9B is a bottom view of another example of the foldable urination device of FIG. 1A in unpackaged position, showing the top part of the funnel of FIG. 1A folding up according to another embodiment of the invention;

FIG. 10A is a top view of one example of a foldable urination device of FIG. 1A in a flattened position, showing the top part of the funnel of FIG. 1A unfolded according to one embodiment of the invention;

FIG. 10B is a bottom view of another example of the disposable and foldable male urination device of FIG. 1 in a flattened position, showing the top part of the funnel of FIG. 1A unfolded according to another embodiment of the invention;

FIG. 11 is a perspective view of another example of the foldable urination device of FIG. 1A in a flattened position, showing a handle of FIG. 1A unfolded according to another embodiment of the invention;

FIG. 12 is a top side view of one example of a foldable urination device of FIG. 1A in an expanded position according to one embodiment of the invention;

FIG. 13A is a perspective view of another example of the foldable urination device of FIG. 1A in an expanded position, showing a flap of FIG. 1A folded into the bottom opening according to another embodiment of the invention;

FIG. 13B is a right side view of another example of the foldable urination device of FIG. 1A in an expanded position, showing a flap of FIG. 1 folded into the bottom opening according to another embodiment of the invention;

FIG. 13C is a right side view of one example of the foldable urination device of FIG. 1A in an expanded position, showing a flap of FIG. 1A folding into the bottom opening according to one embodiment of the invention;

FIG. 14 is a flowchart of a method of an example of operating a urination device having one or more flaps and one or more handles according to one embodiment of the invention;

FIG. 15 is a plan view of a foldable urination device having a single handle;

FIG. 16 is a plan view the foldable urination device of FIG. 15 with the short tabs folded and adhered to the body;

FIG. 17 is a plan view of an unfolded double handle for attachment on the device of FIG. 18;

FIG. 18 is a plan view of an unfolded urination device configured to attached to the double handle of FIG. 17;

FIG. 19 is a plan view of a folded double handle;

FIG. 20 is perspective view of the device of FIG. 15 in a folded state and in a passageway configuration;

FIG. 21 is a plan view of the device of FIG. 15 in a folded state, folded flat along a central fold line;

FIG. 22 is a plan view of the device of FIG. 18 in a folded state, folded flat along a central fold line;

FIG. 23 illustrates the folding and attachment of a double handle to the device of FIG. 22;

FIG. 24 illustrates the folding flat of the device of FIG. 23;

FIG. 25A illustrates the folded state and passageway configuration of the device of FIG. 18;

FIG. 25B illustrates the collapsing of the device of FIG. 25B to a folded flat configuration;

FIG. 26A is a rear view of the device of FIG. 15 in a folded state;

FIG. 26B is a front view of the device of FIG. 15 in a folded state;

FIG. 27 is a plan view illustrating various dimensions and angles forming the urination device of the present invention;

FIG. 28 is perspective view of the urination device of FIG. 27 folded into a passageway configuration by folding a right side tab.

FIG. 29 is perspective view of the urination device of FIG. 27 folded into a passageway configuration by folding a right side tab, with the handle folded up.

FIG. 30 is a perspective view of the device of FIG. 15, in a folded state and a passageway configuration.

FIG. 31 is a side view of the device of FIG. 30 in a flat configuration but folded at the central fold line.

FIG. 32 is left side view of the device of FIG. 15 in a folded passageway configuration.

FIG. 33 is a right side view of the device of FIG. 15 in a folded passageway configuration.

FIG. 34 is a top view of the device of FIG. 15 in a folded passageway configuration.

FIG. 35 is a bottom view of the device of FIG. 15 in a folded passageway configuration.

FIGS. 36-38 illustrate a step by step process of converting the device of FIG. 15 from a flat configuration in FIG. 36 to rotating handle along long tab fold line in FIG. 37 and then forming the device into a folded state in a passageway configuration of FIG. 38.

FIGS. 39-41 illustrate a step by step process of converting the device of FIG. 18 from a flat configuration in FIG. 39 to rotating handle along long tab fold line in FIG. 40 and then forming the device into a folded state in a passageway configuration of FIG. 41.

FIG. 42 is a view similar to FIG. 26A but with central fold line moved down.

FIG. 43 is a left side perspective views of the double handle urination device of FIG. 18 in a folded passageway configuration.

FIG. 44 is a left side perspective views of the double handle urination device of FIG. 18 in a folded passageway configuration.

FIG. 45 illustrates a user operating the foldable urination device.

### DETAILED DESCRIPTION OF THE EMBODIMENTS OF THE INVENTION

The present invention provides an expandable male urination device that helps guide the urine stream for a male with buried penis, hidden penis, penis shrinkage, benign prostatic hypertrophy, cancer of the prostate/bladder, Urinary blockage, Neurogenic bladder dysfunction, or any disease/disorder which may affect the urine stream.

### Expandable Urination Device

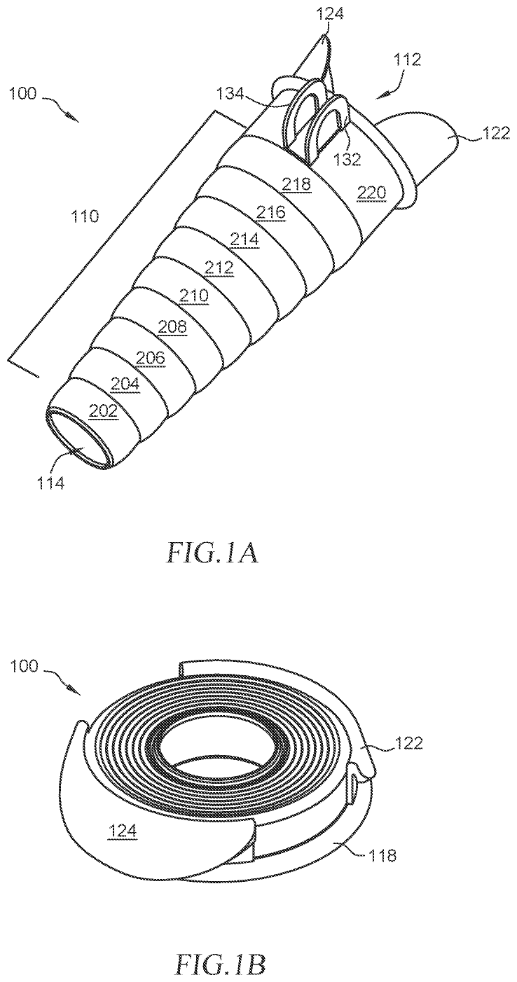

FIG. 1A illustrates a perspective view of one example of a collapsible urination device 100, which is disposable and expandable, in an expanded position. The urination device 100 can also be collapsible into a storage position, as shown later in FIG. 1B, The urination device 100 generally includes a body 110, a first opening 112, a second opening 114, a rim 118, flaps 122, 124, and handles 132, 134. In one example, the body 110 can be in a funnel shape or any other suitable shape.

As another example, the body 110 may include a first ring-shaped element 220 and a number of second ring-shaped elements, such as two or more second ring-shaped elements, three or more second ring-shaped elements, e.g., the second ring-shaped elements 202, 204, 206, 208, 210, 212, 214, 216, 218. In one embodiment, the first ring-shaped element 220 can be larger in size than the second ring-shaped elements. The rim 118 is around the outside of the bottom of the first ring shape element 220.

The urination device 100 is expandable and collapsible and includes a first opening 112, which is large enough for receiving the urine at one end, and a second opening 114 at another end for discharging urine. The flaps 122, 124 form into the circumference of the first opening 112 to prevent urine splashback. The handles 132, 134 of the urination device 100 are provided for holding the urination device 100 as a whole by a user.

To use the urination device 100, the user unfolds the flaps 122, 124 and the body 110 so as to extend the urination device 100 into an expanded position. When using the urination device 100, the user holds the urination device 100 by a hand (or both hands) and uses the flaps 122, 124 the urination device 100 to cover the perineum and/or the penis portions of the user. The handles 132, 134 are adapted to allow the user to hold steady the urination device 100 so that the user can start to void urine.

For example, an individual male can put the urination device 100 onto a penis for guiding the user's urine stream. For example, when a male user is standing in front of the urinal, he can cover the perineum with the urination device 100 by holding the one or more handles 132, 134. When the user starts to void, the urine will flow from the first opening 112 through the body 110 to the second opening 114. The one or more flaps 122, 124 on the sides of the first opening 112 prevent urine splashback from the inside of the urination device 100 and guide splashed urine to the first opening 112. After the male user finishes urinating with the urination device 100 of the present invention, he can take off the urination device 100 and flush it down the toilet or dispose of it.

In one embodiment, the first ring-shaped element 220 is positioned close to the first opening 112. In another embodiment, the first ring-shaped element 220 and the second ring-shaped elements 202, 204, 206, 208, 210, 212, 214, 216, 218 are adapted to be stacked concentrically and stored in a packaged form and extended to an expanded form. Further, the first ring-shaped element 112 is larger in size than the second ring-shaped elements 202, 204, 206, 208, 210, 212, 214, 216, 218, and is located in the outer most location when stacked concentrically.

In some embodiment, the first opening 112 has a size which can cover an average perineum so that the penis can be placed into the urination device 100 easily. For example, the body 110 may have a length ranging from about 5 cm to about 10 cm. In one example, the second opening 114 of the urination device 100 has a diameter of about 5 cm or smaller, such as about 3 cm or smaller, or about 2 cm or smaller. In another example, The first opening 112 of the urination device 100 has a diameter of about 6 cm or smaller, such as about 4 cm or smaller, or about 3 cm or smaller, or about 2 cm or smaller. In one embodiment, the diameter of the second opening 114 can be smaller than the diameter of the first opening 112.

In another embodiment, the flaps 122, 124 forming into the first opening 112 is positioned; one or more handles 132, 134 attached to the first ring-shaped element 220 so that a user of the urination device 100 is able to hold the urination device 100 via the one or more handles 132, 134 for directing a urine liquid away from the user while the user is using the urination device 100.

The urination device 100 can be used in any setting, including home, public facilities, travel, airplane, etc. The device is preferably made of flushable paper or the like and is light weight and easily disposable. This feature allows the device can be easily flushed away by toilets or disposed of at any garbage can, recycling bin or trash container.

The capability of quick unpacking and collapsibility of the urination device 100 offers easy portability and compact storage. This quick packaging profile of the urination device 100 allows for compact packaging and efficient use of shelf space in the retail environment and within coin operated dispenser machines that are typically located within public toilet facilities. The compact profile also allows convenient portability of the disposable and foldable male urination device within a user's bag or wallet and the like.

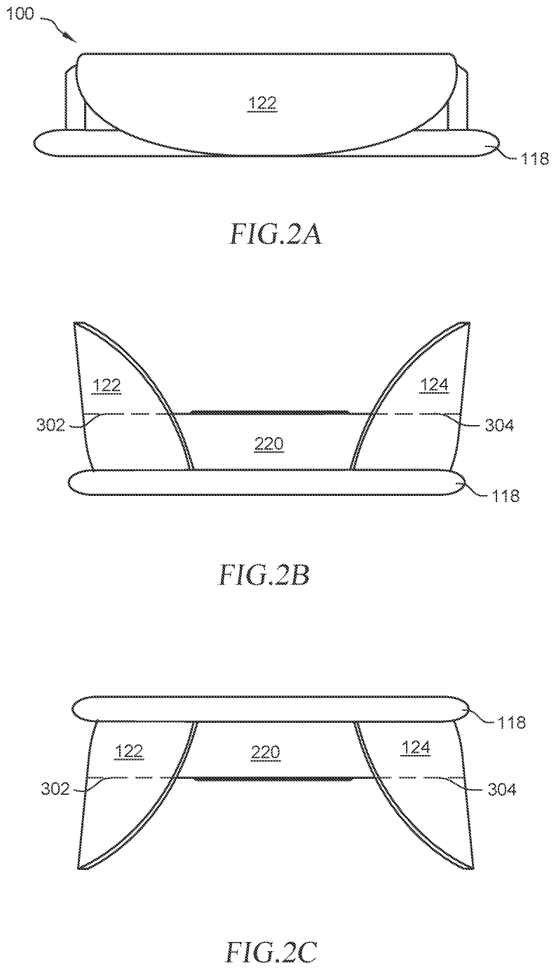

FIG. 1B illustrates a perspective view of another example of the disposable and foldable male urination device in an in packaged position. The rim 118 serves as a base which allows the urination device 100 to be placed on a flat surface. The one or more flaps 122, 124 are folded up for compact storage. As shown in FIG. 2A, the top end of the flap 122 folds down to the rim 118.

FIG. 2B and FIG. 2C illustrate a front view of one example of a disposable and foldable male urination device of FIG. 1B in unpackaged position, showing one or more flaps of FIG. 1B unfolded. A flap folding line 302 and a flap folding line 304 are at the same level as the top surface of the first ring-shaped element 220. The flap 122 expand along the flap folding line 302 and the flap 124 expand along the flap folding line 304.

In still another embodiment, the urination device 100 includes a rim 118 connected to the body 100 and the one or more flaps 122, 124, and one or more handles 132, 134 are adapted to be folded together to be held by the user and unfolded to be stored.

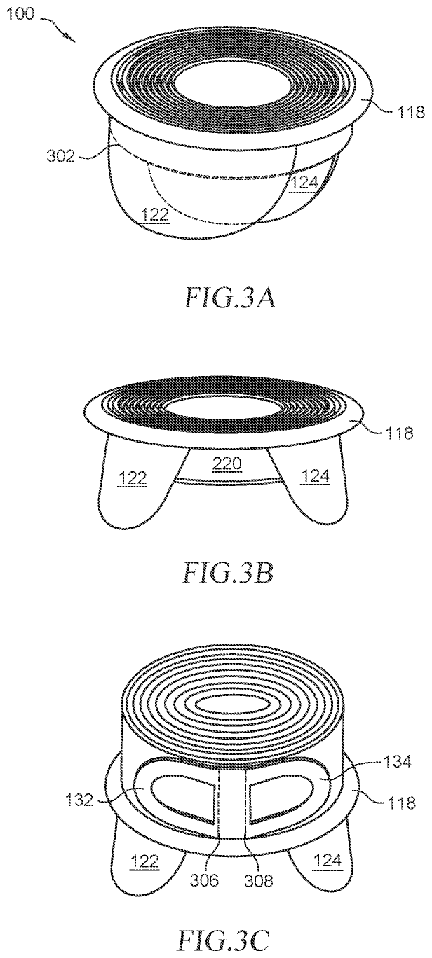

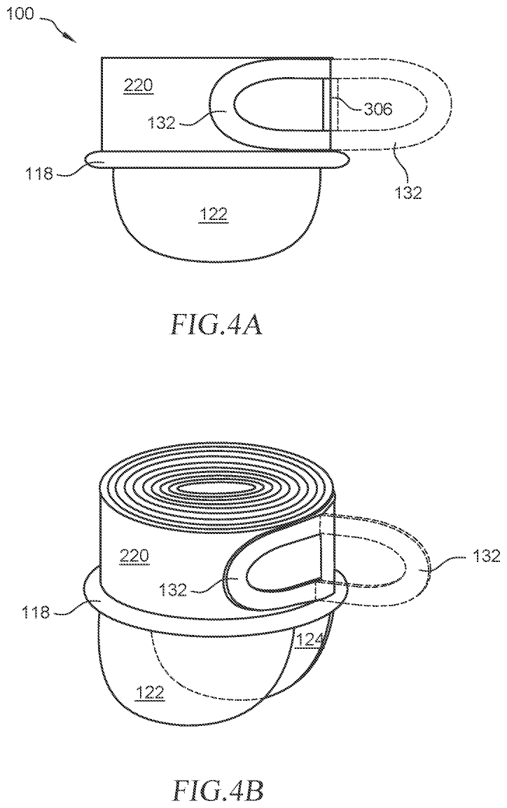

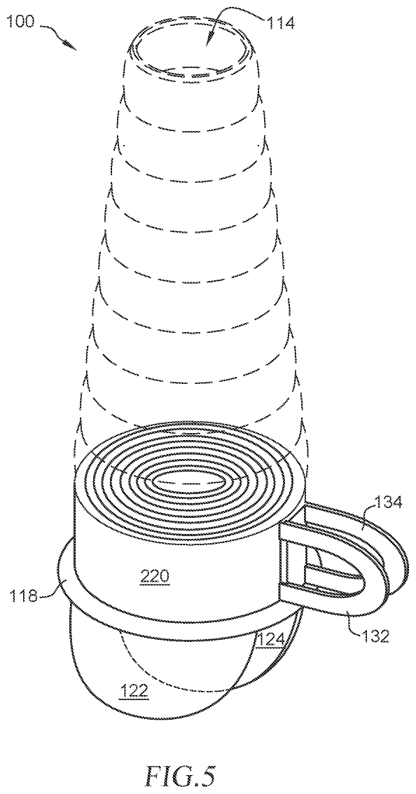

When a male user unpacks the urination device 100, he firstly unfolds the one or more flaps 122, 124 and turns the rim 118 to the top. (e.g., as shown in FIG. 3A and FIG. 3B). Then, the male user holds the rim 118 and presses the first shaped ring element 220 from the bottom. The bottom of the first shaped ring element 220 will move upward to the same position as the rim 118, and the first shape ring element 220 will expand above the rim 118. (e.g., as shown in FIG. 3C) The one or more handles 132, 134 are attached on the side surface of the first shaped ring element 220. The handle 132 can fold up along a handle folding line 306, (e.g., as shown in FIG. 4A) and the handle 134 can fold up along a handle folding line 308.

In yet another embodiment, the urination device 100 includes the body of the urination device 110 is adapted to be folded in at least half of its original size using one or more folding lines 302, 304, 306, 308 as guides, and the one or more flaps 122, 124 are capable of being folded into a packed storage position and being extended in use in an extended shape.

FIG. 4B illustrates a perspective view of another example of the disposable and foldable male urination device of FIG. 1A in a flattened position, showing a handle of FIG. 1A unfolded. After unpacking the urination device 100, the flap 132, 134 can be folded up so as to allow a male user to hold the handles with his thumb and index finger (e.g., as shown in FIG. 5). To extend the urination device 100, the male user can pull the second ring-shaped elements out from the first ring-shaped element 220. The second ring-shaped elements will form the funnel shaped body 110 when the urination device 100 is in an extended position. (e.g., as shown in FIG. 1A)

When using the urination device 100, the user holds the handles 122, 124 and places his penis into the urination device via the first opening 112, and aims the second opening 114 at a urinal or toilet bowl before urinating. When the male user starts urinating, the urine will flow along an inner wall of the urination device and then flows toward the second opening 114 through the funnel shaped body 110. The urine discharged from the second opening 114 will fall into the urinal or toilet bowl. In addition, the male user using the urination device is able to adjust the length of the funnel shaped body 110 of the urination device via the one or more second ringshaped elements to a desirable position.

In another embodiment, the urination device 100 includes the first opening 112 is adapted to receive a urine liquid of the user, and the urine liquid is directed to flow through the body of the urination device 110 and out of the urination device 100 through the second opening 114 to a target location.

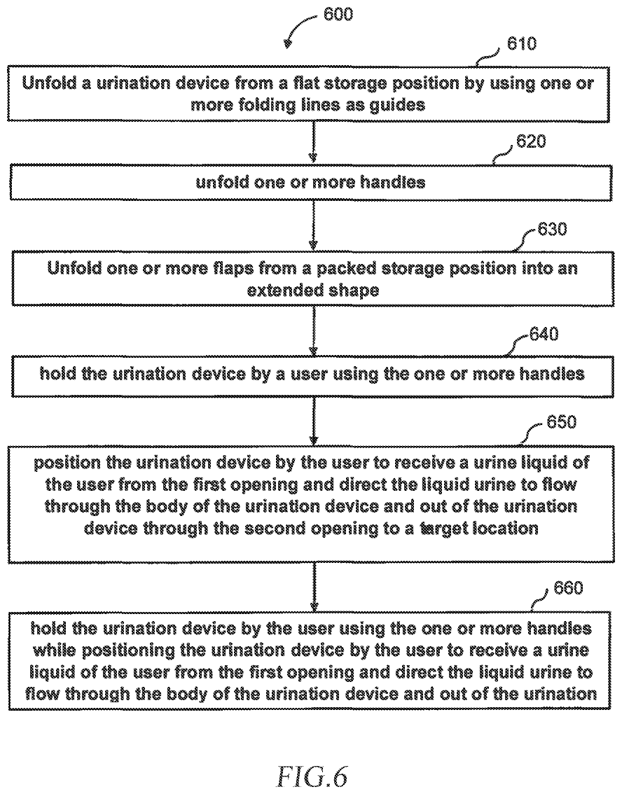

FIG. 6 is a flowchart of an example of a method 600 of using a urination device. The method 600 includes a step 610 of unfolding the urination device from a flat storage position by using one or more folding lines as guides. At step 620, the user using the urination device to unfold the one or more handles. At step 630, the user using the device is able to unfold the one or more flaps from a packed storage position into an extended shape.

At step 640, the user using the urination device is able to hold the urination device by the user using the one or more handles 640. At step 650, the user using the urination device is able to position the urination device by the user to receive a urine liquid of the user from the first opening and direct the liquid urine to flow through the body of the urination device and out of the urination device through the second opening to a target location.

Last but not least, at step 660, the user using the urination device is able to hold the urination device by using the one or more handles while positioning the urination device by the user to receive a urine liquid of the user from the first opening and direct the liquid urine to flow through the body of the urination device and out of the urination device through the second opening to a target location.

Finally, the male user can take off the urination device of the present invention from his perineum after the urination is finished. The male user can simply throw the urination device in a toilet bowl or trash can. The one or more handles can keep the male user's hand away from the remaining urine on the when disposing of the used urination device in the toilet bowl or trash can.

### Foldable Urination Device

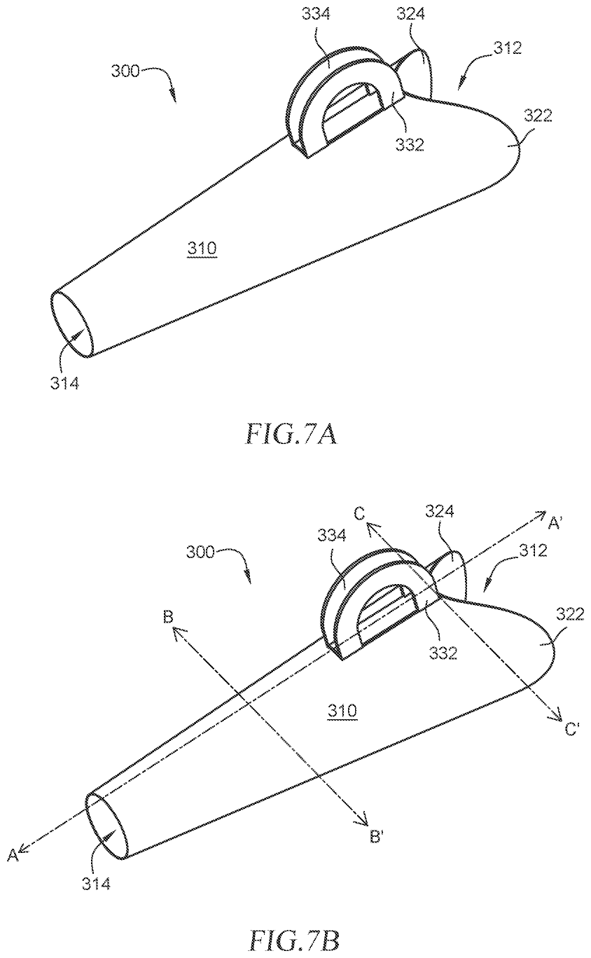

In another embodiment, referring to FIGS. 7-14, a foldable urination device comprises a large opening for receiving the urine at a bottom end and a small opening at a top end for discharging urine. The bottom opening is formed with one or more flaps to prevent urine splashback. The device further comprises a fold away handle for holding at the bottom and a foldaway funnel at the top.

To use the device, a user unfolds the flaps and the funnel so as to extend the disposable male urination device into an extended position. When using the male urination device, the user holds the device with a hand and cover the device on the penis then start to void. The handle allows the user to hold the device steady. [0027] The device can be used in any setting, including home, public facilities, travel, airplane, etc. The device is preferably made of flushable paper or the like and is light weight and easily disposable. This feature allows the device can be easily flushed away by toilets or disposed of at any garbage can, recycling bin or trash container.

The capability of quick unpacking and collapsibility of the device offers easy portability and compact storage. This quick packaging profile of the device allows for compact packaging and efficient use of shelf space in the retail environment and within coin operated dispenser machines that are typically located within public toilet facilities. The compact profile also allows convenient portability of the disposable and foldable male urination device within a user's bag or wallet and the like.

FIG. 7A and FIG. 7B illustrates a perspective view of one example of a foldable urination device 300 in an expanded position, which generally includes a funnel shaped body 310, comprising a first opening 312, a second opening 314, one or more flaps 322, 324, and one or more handles 332, 334. The one or more flaps 322, 324 are on the sides of the first opening 312. The one or more handles 332, 334 are attached on the top side of the body 310. Furthermore, a handle folding line A-A′ is provided from the center of the first opening 312 to the center of the second opening 314. A body folding line B-B′ is provided at the center of the body 310. A flap folding line C-C′ is provided between the one or more flaps 322,324 and the one or more handles 332, 334. The handle folding line A-A′, body folding line B-B′ and flap folding line C-C′ are all represented by a dash-dot-dash line.

For example, an individual male can put the foldable urination device 300 onto a penis for guiding urine stream. For example, when a male user is standing in front of the urinal, he can cover the perineum with the foldable urination device 300 by holding the one or more handles 332, 334. When the user starts to void, the urine will flow from the first opening 312 through the body 310 to the second opening 314. The one or more flaps 322, 324 on the sides of the first opening 312 prevent urine splashback from the inside of the urination device 300 and guide splashed urine to the first opening 312. After the male user finishes urinating with the urination device 300 of the present invention, he can take off the urination device 300 and flush it down the toilet or dispose of it.

In one embodiment, a body 310 being shaped in a rounded funnel shape when the body 310 is unfolded and expanded in use, the body 310 includes a first opening 312, one or more flaps 322, 324 forming into the first opening 312, and a second opening 314 smaller than the first opening 312, and one or more handles 332, 334 attached to a portion of the body 310 close to the first opening 312 so that a user of the urination device 300 is able to hold the urination device 300 via the one or more handles 332, 334 for directing a urine liquid away from the user while the user is using the urination device 300.

In some embodiment, the first opening 312 has a size which can cover an average perineum so that the penis can be placed into the urination device 300 easily. In some embodiments, the body 310 has a length more than 30 cm. According to the male urination device of the present invention, the second opening 314 has a diameter of 2 cm and is smaller than the first opening 312.

FIG. 8 illustrates a perspective view of another example of the foldable urination device in packaged position. The handle 332 fits on the surface of the folded body 310 for packaging. The packaged urination device 300 has a compact size which is reduced to less than one quarter the size of the urination device 300. The packaging allows a male user to carry the urination device 300 in his wallet, pants pocket, jacket pocket or a handbag.

In another embodiment, the handles 332, 334 are adapted to be folded together to be held by the user and unfolded to be stored, and the body 310 are adapted to be folded in at least half of its original size using the folding lines A-A′, BB′, and C-C′ as guides.

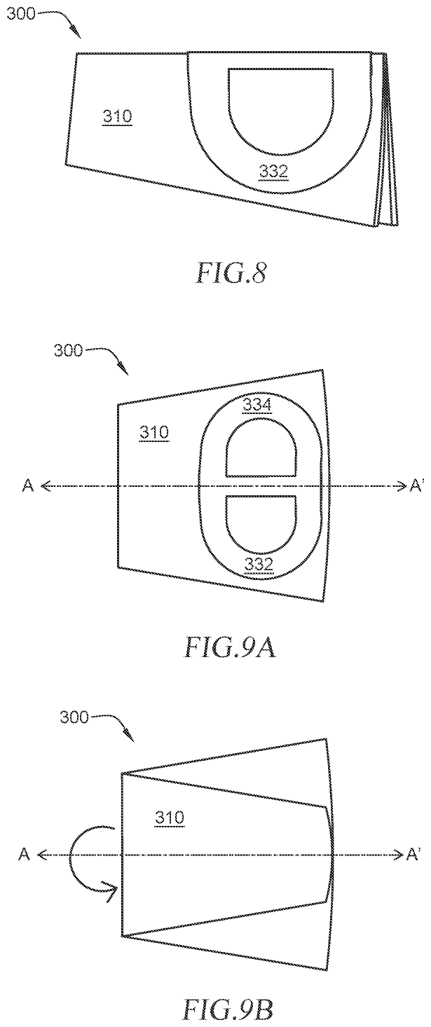

FIG. 9A and FIG. 9B illustrate a top and bottom view of one example of a disposable and foldable male urination device of FIG. 7A in unpackaged position, showing the top part of the funnel of FIG. 7A unfolded. The body 310 expands along a handle folding line A-A′. (defined as from a center top to a center end of the body 310) The handles 332, 334 are attached on the top surface of body 310 and stay unfolded on the sides of the handle folding line A-A′.

In still another embodiment, body 310 is adapted to be folded so as to be in a flat storage position, and the body 310 is adapted to be unfolded and expanded into a rounded funnel shape by using the handles 332, 334 and the folding lines AA′, B-B′, and C-C′ as guides.

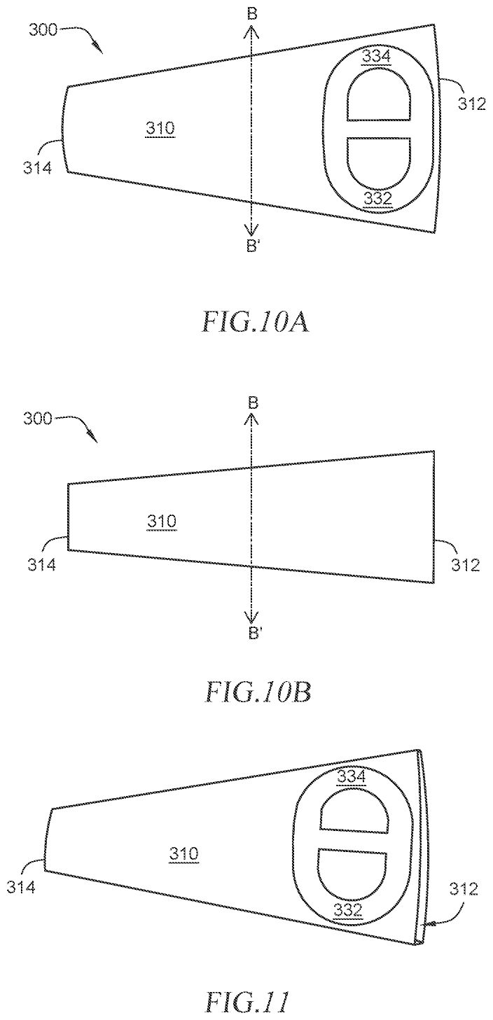

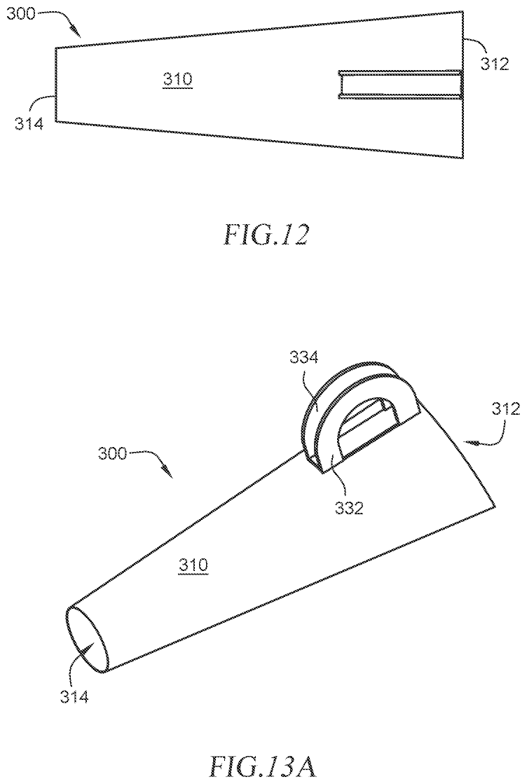

For example, when a user unpacks the urination device 300, he firstly unfolds the two sides of the body 310. (e.g., as shown in FIG. 9A and FIG. 9B) and then unfolds the top part of the funnel shaped body 310 along a body folding line B-B′ (e.g., as shown in FIGS. 10A and 10B). To expand the urination device 300, the user can hold the body 310 and expand the first 312 from the flatten position to expanded position. (e.g., as shown in FIG. 11) The handles 332, 334 can fold up along the handle folding lines A-A′. (e.g., as shown in FIG. 12)

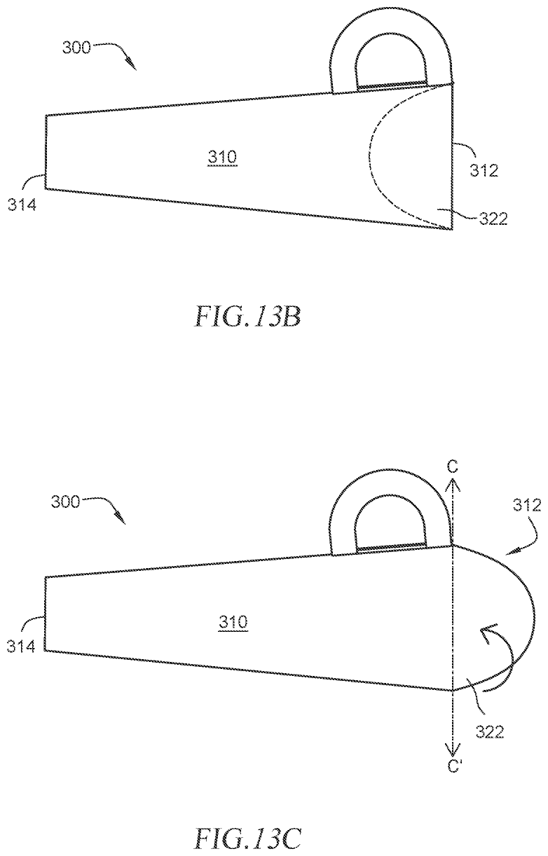

FIG. 13A illustrates a perspective view of another example of the foldable urination device of FIG. 7A in an expanded position, showing a flap of FIG. 7A folded into the bottom opening. After unpacking the urination device 300, the flap 332, 334 can be folded up so as to allow a user to hold the handles with his thumb and index finger. To complete the expanded position of the urination device, the user can unfold the flap 322, 324 along a flap folding line C-C′. (e.g., as shown in FIGS. 7B and 7C)

For another example, when using the urination device, the user holds the handles 322, 324 and places his penis into the urination device via the first opening 312, and aims the second opening 314 at a urinal or toilet bowl before urinating. When the user starts urinating, the urine will flow along an inner wall of the urination device and then flows toward the second opening 314 through the funnel shaped body 310. The urine discharged from the second opening 314 will fall into the urinal or toilet bowl.

In another embodiment, a body 310 being shaped in a rounded funnel shape when the body 310 is unfolded and expanded in use, the body 310 includes a first opening 312, the flaps 322, 324 forming into the first opening 312; and a second opening 314 smaller than the first opening 312 and the handles 332, 334 being attached to a portion of the body 310 close to the first opening 312 and adapted to direct a urine liquid away from a user while the user is using the urination device 300, wherein the handles 332, 334 are adapted to be folded together to be held by the user and unfolded to be stored.

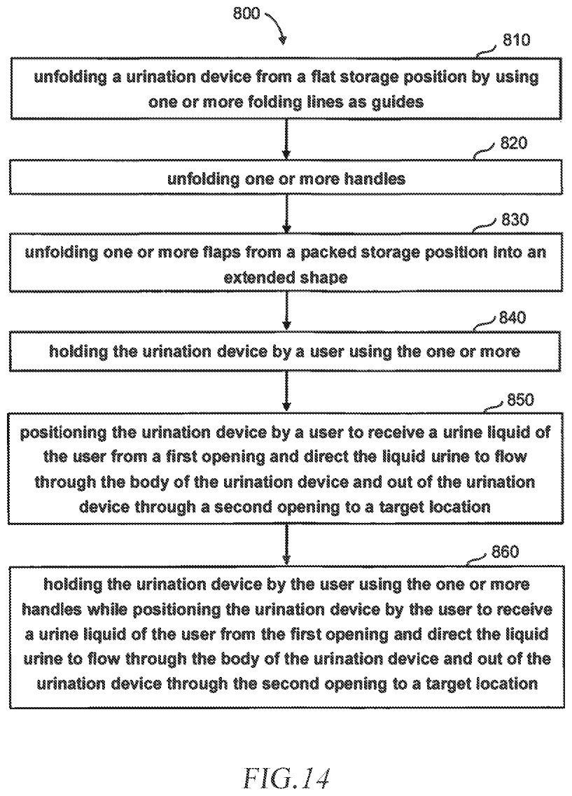

FIG. 14 is a flowchart of a method 800 of an example of operating a urination device having the flaps and the handles according to one embodiment of the invention.

At step 810, the user using the urination device is able to unfold the urination device from a flat storage position by using the folding lines as guides.

At step 820, the user using the urination device is able to unfold the handles And at step 830, the user using the urination device is able to unfold the flaps from a packed storage position into an extended shape.

At step 840, the user using the urination device is able to hold the urination device by the user using the handles.

At step 850, the user using the urination device is able to position the urination device by the user to receive a urine liquid of the user from the first opening and direct the liquid urine to flow through the body of the urination device and out of the urination device through the second opening to a target location.

Last but not least, at step 860, the user using the urination device is able to hold the urination device by the user using the handles while positioning the urination device by the user to receive a urine liquid of the user from the first opening and direct the liquid urine to flow through the body of the urination device and out of the urination device through the second opening to a target location.

Finally, the user can take off the urination device of the present invention from his perineum after the urination is finished. The user can simply throw the urination device in a toilet bowl or trash can. The handles can keep the user's hand away from the remaining urine on the when disposing of the used urination device in the toilet bowl or trash can.

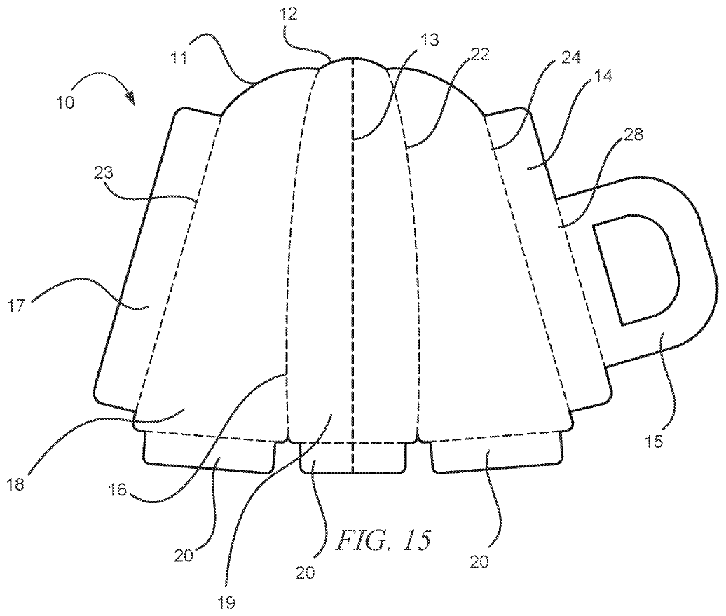

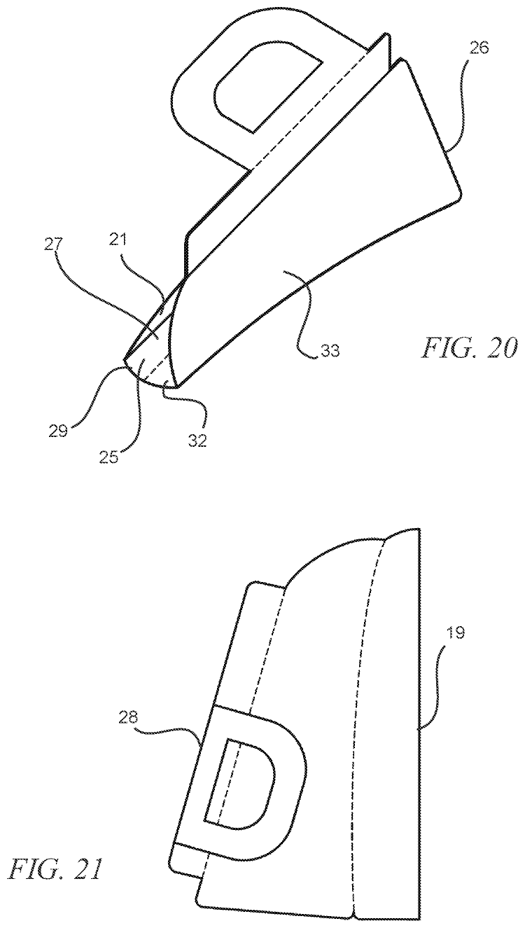

In another embodiment, referring to FIG. 15, a foldable urination device, for deflecting urine away from a user, comprises a body 19 having a central fold line 13, left 16 and right 22 curved fold lines and left 23 and right 24 side fold lines. Preferably, the left and right curved fold lines and the left and right side fold lines are symmetrically disposed on either side of the central fold line. Fold lines are formed by scoring the body, which is preferably made of paper, and which allows biases the body to be folded along the scored lines. A left long tab 17 is connected to a left side of the body at the left side 23 fold line. A right long tab 14 is connected to a right side of the body at the right side 24 fold line. The left curved fold line 16 and the left side fold line 23 are disposed on the left side of the body, and the right curved fold line 22 and the right side fold line 24 are disposed on a right side of the body. The central fold line 13 is disposed between the left and right curved fold lines and side fold lines. Left and right side fold lines 23, 24 are straight lines and left and right curved fold lines 16, 22 are gently curved with large radii of curvature. When the foldable urination device is folded along the curved and side fold lines as illustrated in FIG. 20, the foldable urination device forms a curved passageway having an entrance 26 and an exit 25. The curvature of the curved fold lines are greater (with smaller radii of curvature) near the top part of FIG. 15 than the bottom part of FIG. 15 to generate more of a slope near the exit of the device when the device is formed into a passageway 27. In its unfolded state, the urination device is a flat sheet. As illustrated in FIG. 45, a user of the urination device is able to hold the urination device directing a urine stream away from the user.

Continuing with FIG. 15, a handle 15 is attached to the right side long tab 14 along right side long tab fold line 28, permitting the handle to be pivotable with respect to the urination device. Optionally, the handle 15 may be attached to the left side long tab 14. The body 19 is adapted to be folded in at least half of its original size using one or more folding lines as guides, or preferably, along the central fold line 13, for example, as illustrated in FIG. 21. When folded as in FIG. 21, the device is folded into two flat layers resting against each other for efficient packaging and transport.

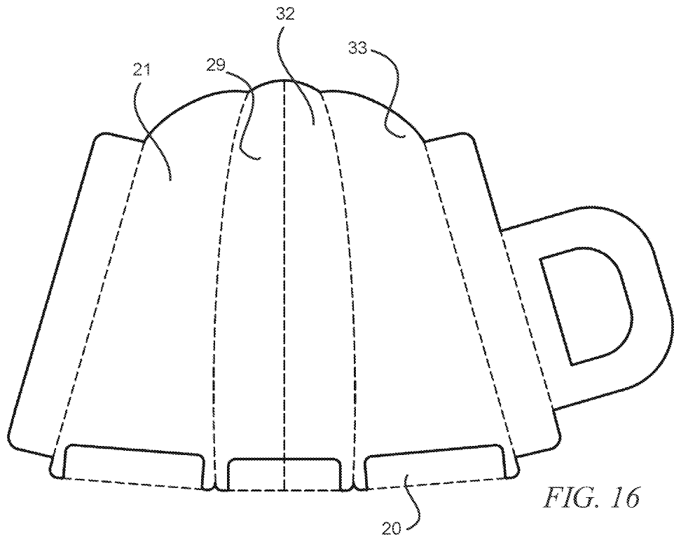

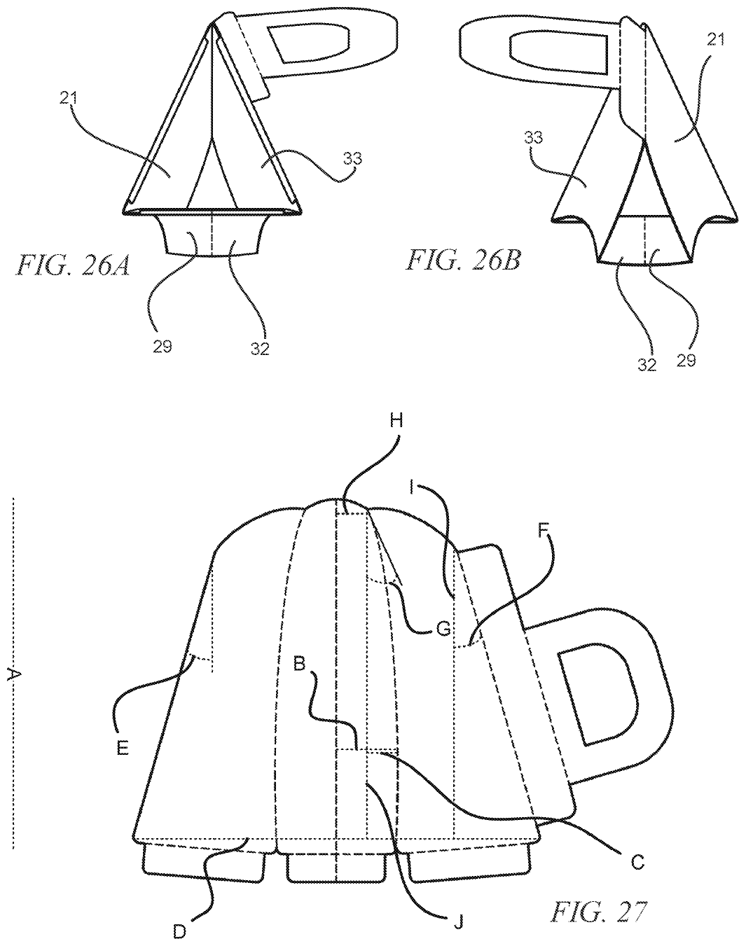

Referring to FIG. 15, the urination device has top, bottom, left and right perimeter edges. The top perimeter edge is formed from the arcuate edges, two curved sections on either side of a central curved edge, the central curved edge having greater curvature than the others. The bottom perimeter edge is generally straight. The bottom perimeter edge has short tabs 20 attached thereto. As illustrated in FIG. 16, the short tabs 20 are attached to the body at fold lines and then folded along the fold lines and adhered to the body via their planar surfaces. The short tabs 20 are formed around the entrance serve to protect a user against sharp edges when the entrance 26 to the device when the device is in a folded state and in use. When in a folded state, the device comprises four sides, and has at two curved sides. Because the bottom edges are longer than that the top arcuate edges, when in a folded state, the entrance 26 is larger than the exit 25. When the urination device is in a folded state, the left and right long tabs 14, 17 are adhered to each other's surfaces as illustrated in FIG. 20. Surface sections 27, 29, 32 and 33 are formed as illustrated in FIGS. 20 and 26, which are formed and bounded by curved, side and central fold lines 13, 16, 22, 23.

In yet a further embodiment, referring to FIG. 27, the configuration of the device, distances and angles of the various edges and fold lines, may be defined by parameters A through J. In one embodiment, distance A is 7.0 inches but may vary +/−1.0 inches from 6 to 8 inches. In general, the values stated herein may vary up or down by at least by 15%-30% without departing from the general invention. Distance B is the greatest perpendicular distance from central fold line 13 to right side curved fold line 22 which is 1.2 inches and may vary +/−0.2 inches from 1.0 to 1.4 inches. C measures a port of distance B, however for the sake of clarity, the to distances are drawn separately. However, C is the greatest perpendicular distance from distance B to right side curved fold line 22 which is 0.6 inches and may vary +/−0.12. D is generally the greatest width of the device and is 7.9 inches and may vary +/−1.6 inches. E and F are the angels side edges make with a vertical line, and are each 16.2 degrees and may vary +/−5 degrees. Since the device is generally symmetric, the E and F should be the same. G is an angle right curved line makes with vertical line J. Due to symmetry, left curved line forms the same angle G, which is 25.9 degrees+/−6 degrees. Distance H is the same value as distance B. Distance I is 5.5 inches and may vary +/−1.4 inches. Distance J is 6.4 inches and may vary +/−1.5 inches.

In another embodiment, referring to FIG. 27, A is 10.0 inches but may vary +/−2.0 inches from 8 to 12 inches. Distance B is 1.4 inches and may vary +/−0.3 inches from 1.1 to 1.7 inches. C measures a port of distance B, however for the sake of clarity, the to distances are drawn separately. However, C is the greatest perpendicular distance from distance B to right side curved fold line 22 which is 0.6 inches and may vary +/−0.12. D is generally the greatest width of the device and is 7.9 inches and may vary +/−1.6 inches. E and F are the angels side edges make with a vertical line, and are each 16.2 degrees and may vary +/−5 degrees. Since the device is generally symmetric, the E and F should be the same. G is an angle right curved line makes with vertical line J. Due to symmetry, left curved line forms the same angle G, which is 25.9 degrees+/−6 degrees. Distance H is the same value as distance B. Distance I is 5.5 inches and may vary +/−1.4 inches. Distance J is 6.4 inches and may vary +/−1.5 inches.

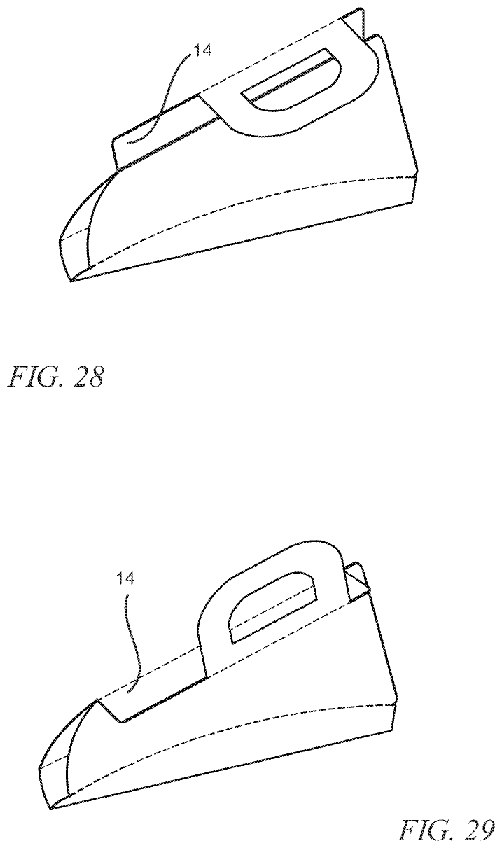

In FIG. 27 the left long tab is no present, but the dimension provided would apply equally to FIG. 15 or FIG. 18 for example. FIGS. 28 and 29 illustrate an example where the device of FIG. 27 can be folded into a passageway configuration by folding right side tab 14 onto and adhering it to a rear surface of the left side of the body.

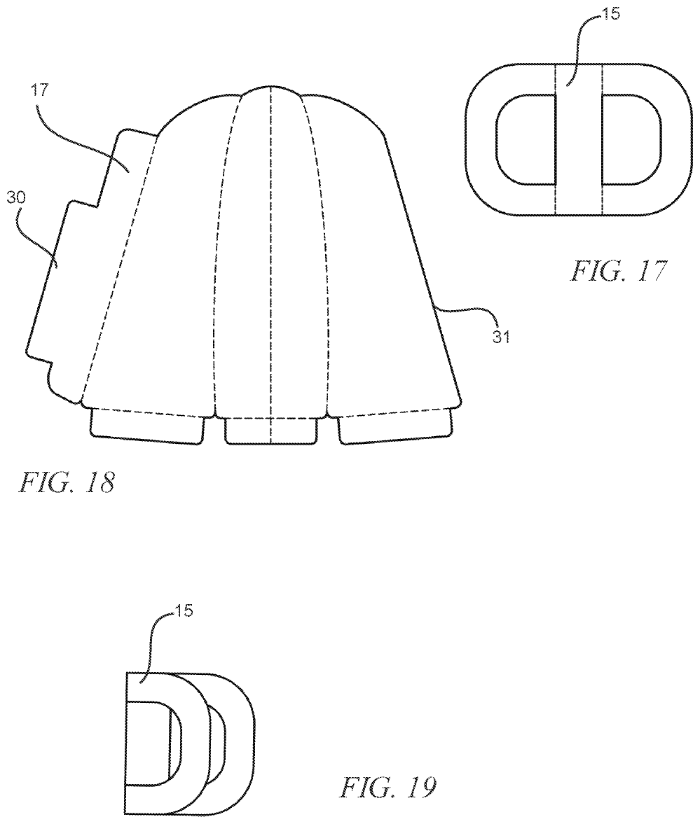

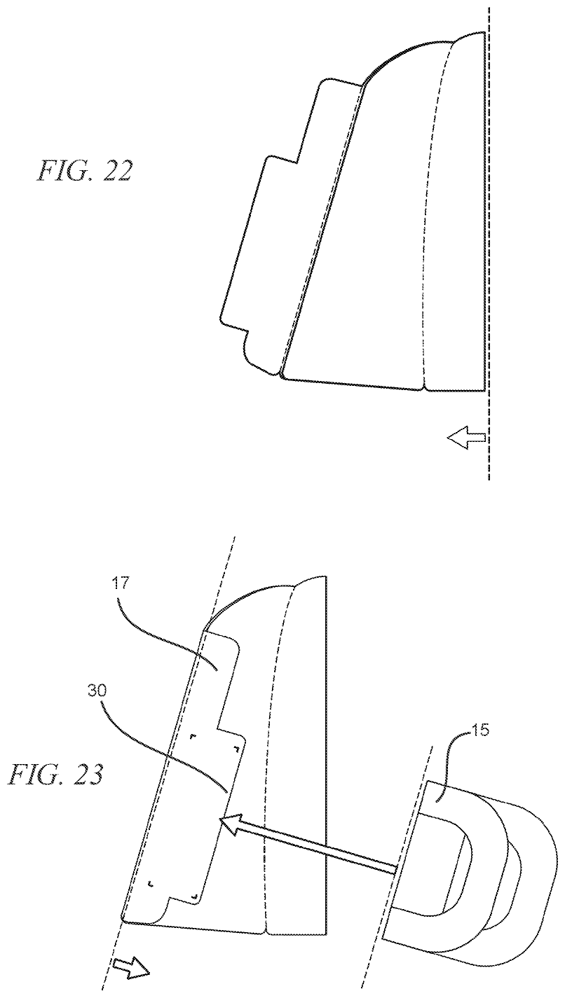

In a further embodiment, the device has only one long tab attached either the left side or the right side of the body. In one example, referring to FIG. 18, the device has only a left long tab 17 attached to a left side of the device. The device has only a left side fold line 16 and no right side fold line due to the absence of a right long tab. Instead, the device has a right side edge 31. When the urination device is in a folded state, the left tab is adhered to the right side of the body as illustrated in FIG. 23. Optionally, the urination device comprises a minor tab 30 connected to the left long tab, for allowing for greater surface area to which a handle 15 can be adhered to. The step of adhering left long tab to the right side of the body is illustrated in FIGS. 22 and 23. The body is folded at central fold line 13 in FIG. 22. Next, left long tab 17 is folded and adhered to the surface of the right side of the body in FIG. 23, using glue or adhesive, for example. Next, handle 15 may be attached to the left long tab 17. Optionally, the handle is attached at the minor tab 30 which forms a wide area for attachment. Optionally, the handle may be double handle 15 as illustrated in FIGS. 17, 19 and 23-25.

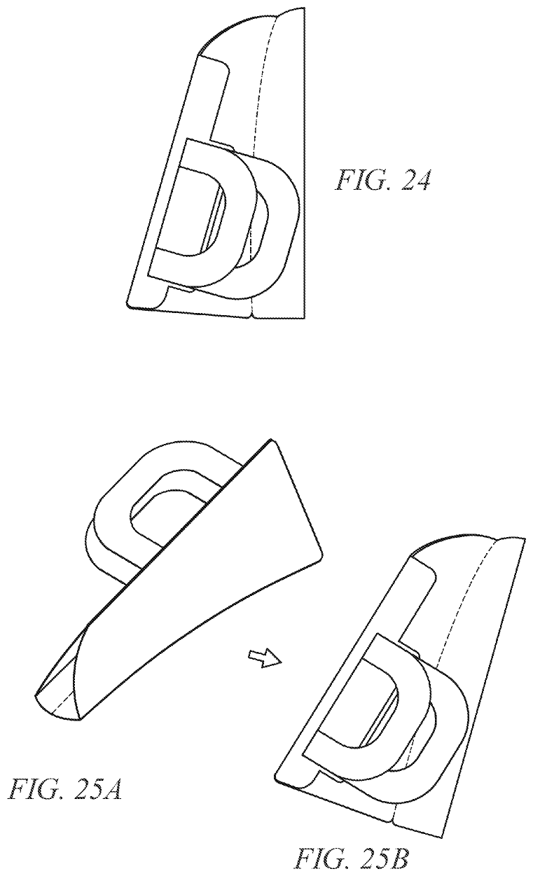

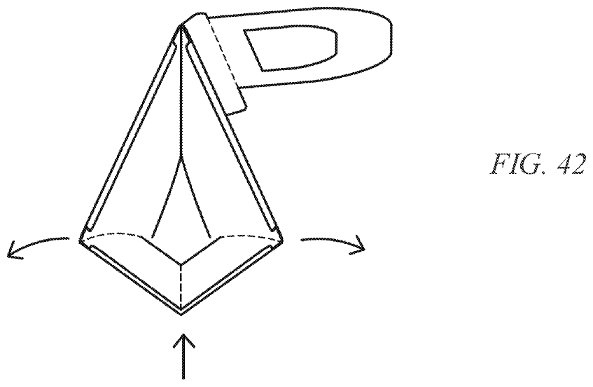

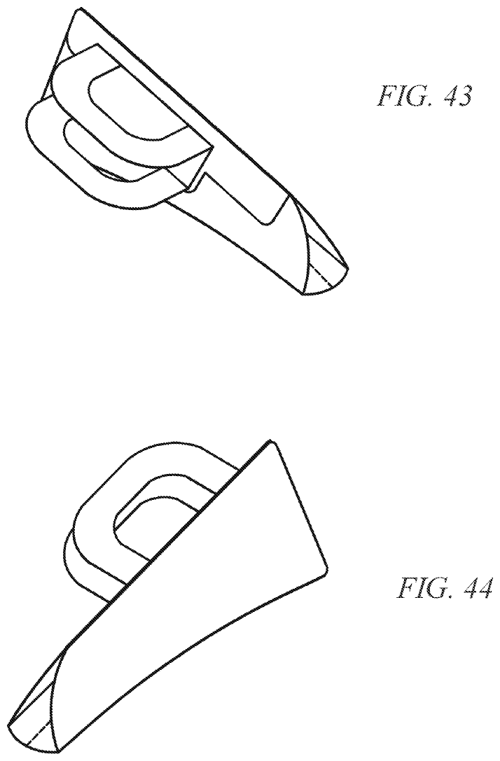

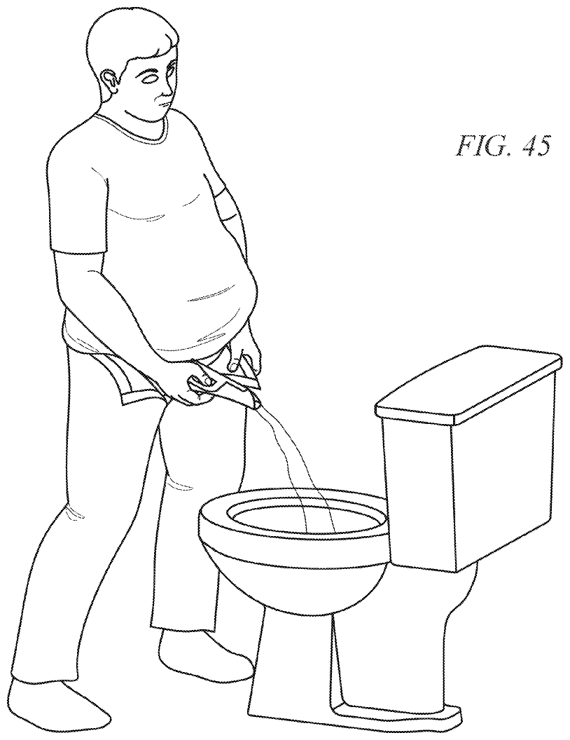

FIGS. 23-25 illustrate how the device may be folded flat along fold lines and having a double handle. FIGS. 25A and 25B illustrate the device converting from a passageway configuration (FIG. 25A) to a flat configuration (FIG. 25B) and having a double handle FIGS. 39-41 illustrate a step by step process of converting the double handle device of FIGS. 18 and 19 from a flat configuration in FIG. 39 to rotating handle 15 along long tab fold line 28 in FIG. 40 and then spreading apart sections 21 and 33 and bringing down central fold line 13 to form a folded state in a passageway configuration of FIG. 41. FIGS. 43 and 44 are respective left and right side perspective views of the double handle urination device of FIG. 18 in a folded passageway configuration.

FIG. 26A illustrates a rear view of the device of FIG. 15, showing the entrance 26 and surface actions 21, 29, 32 and 33, while FIG. 26B illustrates a front view of the device of FIG. 15, showing the exit 25.

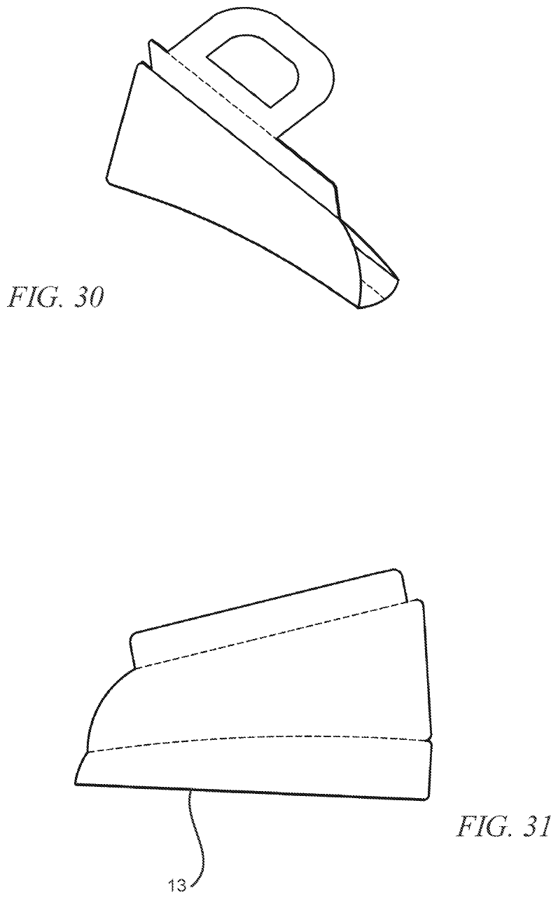

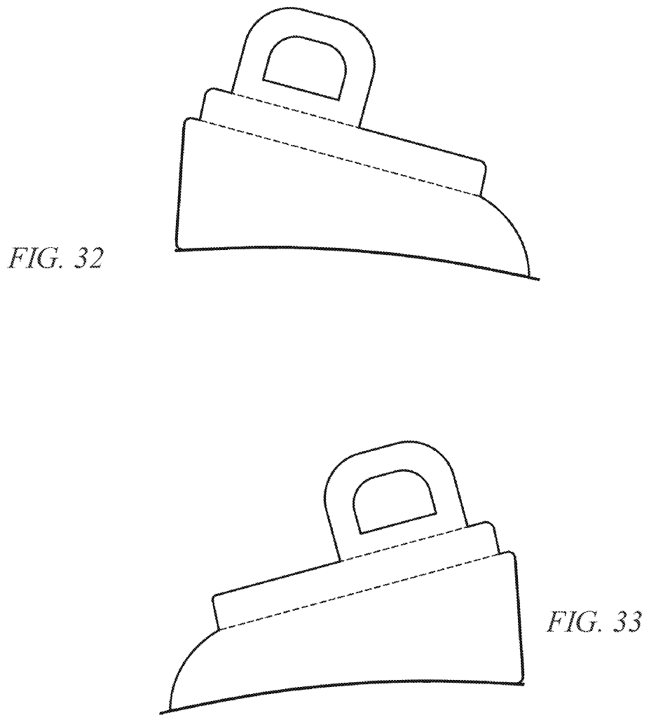

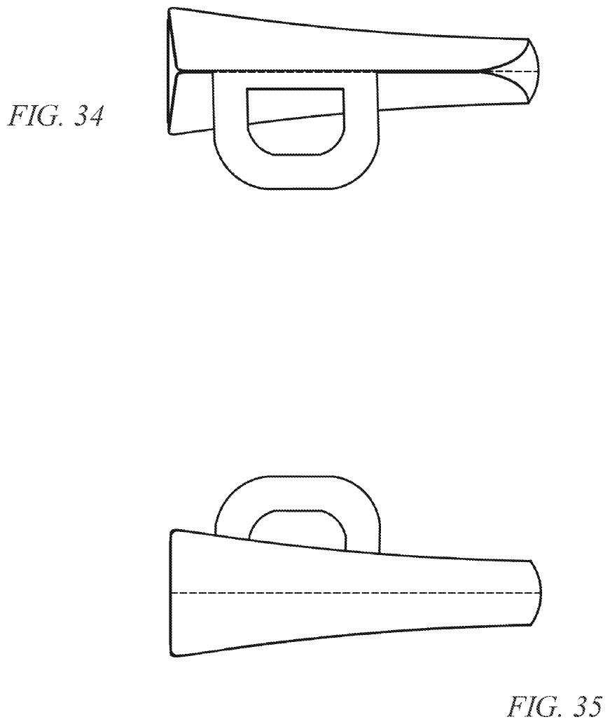

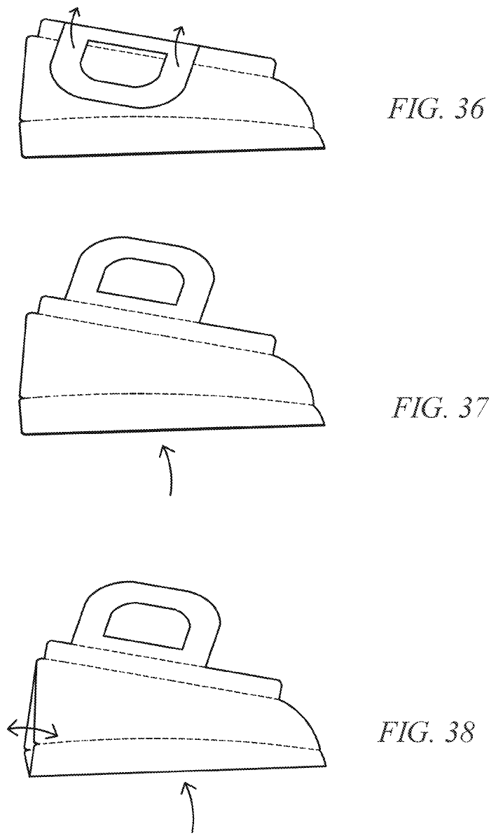

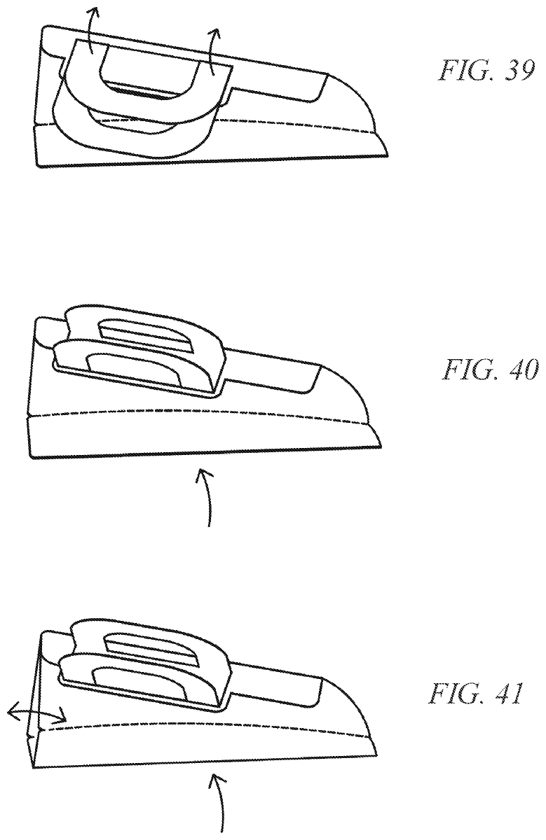

FIG. 30 illustrates a perspective view of the device of FIG. 15, in a folded state and a passageway configuration. FIG. 31 illustrates a side view of the device of FIG. 30 in a flat configuration but folded at the central fold line 13. FIGS. 32 and 33 illustrate left and right side views of the device in a folded passageway configuration. FIGS. 34 and 35 illustrate respective top and bottom views of the device in a folded passageway configuration. FIGS. 35-36 illustrate a step by step process of converting the device from a flat configuration in FIG. 36 to rotating handle 15 along long tab fold line 28 in FIG. 37 and then spreading apart sections 21 and 33 and bringing down central fold line 13 to form a folded state in a passageway configuration. FIG. 42 is a view similar to FIG. 26A but with central fold line 13 moved down. While the embodiments disclosed illustrate a right-handed urination device, it would be readily apparent to those skilled in the art that left-handed or mirror image versions of any embodiment herein described of the device may be constructed based on the principles of the invention.

What is claimed is:

1. A foldable urination device, comprising:
a body having a central fold line, left and right curved fold lines and a left side fold line;
a left long tab connected to a left side of the body along the left side fold line;
wherein the left curved fold line and the left side fold lines are disposed on the left side of the body, and the right curved fold line, wherein the central fold line is disposed between the left and right curved fold lines;
wherein when the foldable urination device is folded along the curved and side fold lines, the foldable urination device forms a curved passageway having an entrance and an exit,
wherein a user of the urination device is able to hold the urination device directing a urine stream away from the user.

2. The urination device of claim 1, further comprising a right fold line disposed on a right side of the body and a right long tab connected to the right side of the body along the right fold line.

3. The urination device of claim 2, wherein the body is adapted to be folded in at least half of its original size using one or more folding lines as guides.

4. The urination device of claim 2, wherein the urination device comprises a flat sheet and the fold lines are scored lines.

5. The urination device of claim 2, further comprising top, bottom, left and right perimeter edges, wherein the top perimeter edge is an arcuate edge.

6. The urination device of claim 2, further comprising top, bottom, left and right perimeter edges, wherein the bottom perimeter edge comprises short tabs attached to the bottom perimeter edge.

7. The urination device of claim 2, wherein in a folded state, the device comprises four edges having at least one curved edge.

8. The urination device of claim 2, wherein in a folded state, the entrance is larger than the exit.

9. The urination device of claim 1, further comprising a minor tab connected to the left long tab.

10. The urination device of claim 9, when the urination device is in a folded state, the left long tab is adhered to the right side of the body.

11. The urination device of claim 2, further comprising a handle attached to one of the long tabs and the long tabs being folded along a side fold line.

12. The urination device of claim 2, wherein the left and right long tabs are attached to each other by their surfaces.

13. The urination device of claim 6, wherein a surface of a short tab is adhered to the body.

14. The urination device of claim 2, wherein the left and right curved fold lines and the left and right side fold lines are symmetrically disposed on either side of the central fold line.

15. A foldable urination device, comprising:
a body having left and right curved fold lines and left and right side fold lines;
left and right long tabs connected to respective left and right sides of the body;
wherein the left curved fold line and the left side fold lines are disposed on the left side of the body, and the right curved fold line and the right side fold line are disposed on a right side of the body;
wherein when the foldable urination device is folded along the curved and side fold lines, the foldable urination device forms a curved passageway having an entrance and an exit,
wherein a user of the urination device is able to hold the urination device directing a urine stream away from the user.

16. The foldable urination device of claim 15, wherein the device is folded and the left and right tabs are adhered to each other.

17. The foldable urination device of claim 15, further comprising a central fold line disposed down a middle of the body.

18. The foldable urination device of claim 15, further comprising a handle attached to one of the tabs and wherein the device is folded into two flat layers resting against each other.

19. A foldable urination device, comprising:
a body having left and right curved fold lines and left and right side fold lines;
left and right long tabs connected to respective left and right sides of the body;
wherein the left curved fold line and the left side fold lines are disposed on the left side of the body, and the right curved fold line and the right side fold line are disposed on a right side of the body, wherein the central fold line is disposed between the left and right curved fold lines and side fold lines;
wherein when the foldable urination device is folded along the curved and side fold lines, the foldable urination device forms a curved passageway having an entrance and an exit;
wherein the curved fold line has greater curvature on a top part of the body and lesser curvature on a bottom part of the body;
wherein the left long tab is adhered to the right side of the body; and
wherein a user of the urination device is able to hold the urination device directing a urine stream away from the user.

20. The foldable urination device of claim 19, further comprising a folded handle attached to the left long tab.

21.-60. (canceled)
